(19) United States
(12) Patent Application Publication
(10) Pub. No.: US 2025/0248612 A1
(43) Pub. Date: Aug. 7, 2025

(54) PHOTOPLETHYSMOGRAPHY SENSING MODULE, SYSTEMS AND DEVICES THEREOF

(71) Applicant: Casana Care, Inc., Rochester, NY (US)

(72) Inventors: David A. BORKHOLDER, Canandaigua, NY (US); Nicholas Joseph CONN, Fairport, NY (US); Matthew WELLMAN, Pittsford, NY (US); David YOUNG, Webster, NY (US)

(21) Appl. No.: 19/091,641

(22) Filed: Mar. 26, 2025

Related U.S. Application Data
(63) Continuation of application No. PCT/US2023/075553, filed on Sep. 29, 2023.
(60) Provisional application No. 63/412,074, filed on Sep. 30, 2022.

(51) Int. Cl. A61B 5/026; A61B 5/00

(52) U.S. Cl. CPC A61B 5/0261; A61B 5/6887; A61B 2560/0462; A61B 2562/0233; A61B 2562/04; A61B 2562/185; A61B 2562/247

(57) ABSTRACT

Systems, devices, and methods are disclosed herein for monitoring physiological data of subjects seated on a toilet, including systems, devices, and methods for monitoring light signals or readings on a toilet seat. In some embodiments, systems, devices, and methods disclosed herein include a set of sensors integrated into a seat (e.g., of a toilet), with the sensors being configured to measure multiple light signals associated with unique optical paths across a portion of tissue of an individual seated on the seat.

### CROSS REFERENCE TO RELATED APPLICATIONS

This application is a continuation of International Patent Application No. PCT/US2023/075553, entitled “Photoplethysmography Sensing Module, Systems and Devices Thereof,” filed Sep. 29, 2023, which claims priority to and the benefit of U.S. Provisional Patent Application No. 63/412,074, entitled “Photoplethysmography Sensing Module, Systems and Devices Thereof,” filed Sep. 30, 2022, the disclosure of which are incorporated by reference herein in their entireties.

### TECHNICAL FIELD

The embodiments described herein related generally to health monitoring systems, and more particularly to systems, devices and methods for monitoring physiological characteristics of seated subjects, including systems and methods for monitoring volumetric changes in blood circulation of the subjects.

### BACKGROUND

Patient health monitoring is an important tool in tracking physiological conditions of patients and to provide early warnings or guidance to individuals and healthcare providers in cases of patient health deterioration. Oftentimes, patient monitoring is obtrusive and requires individuals to actively wear certain devices or change their routine to be able to measure certain vital signs or characteristics of the patient. Unobtrusive systems for monitoring individuals are also limited and can provide inaccurate results. Therefore, there exists a need to develop more accurate approaches to monitoring individuals through unobtrusive means.

### SUMMARY

Systems, devices, and methods are described here for monitoring data (e.g., electromagnetic and/or optical signals) reflected from individuals seated on a toilet.

In some embodiments, an apparatus comprises a plurality of light sources disposed in a seat of a toilet and configured to generate electromagnetic radiation when a subject is seated on the toilet; a sensor disposed on the seat of the toilet, the sensor configured to measure electromagnetic radiation generated by the plurality of light sources and reflected by tissue of the subject when the subject is seated on the toilet; and a partitioning element disposed about the plurality of light sources and the sensor, the partitioning element configured to direct the electromagnetic radiation being generated by the plurality of light sources toward the tissue of the subject seated on the toilet. The apparatus also comprises a processor operatively coupled to the plurality of light sources and the sensor, the processor configured to receive signals from the sensor, the signals associated with different paths between at least one light source from the plurality of light sources and the sensor, and process the signals to determine a physiological parameter of the subject.

### BRIEF DESCRIPTION OF THE DRAWINGS

FIG. 1A is a schematic illustration of a photoplethysmography (PPG) sensing device for measuring light signals reflected from a subject seated on a toilet, according to an embodiment.

FIG. 1B is a schematic illustration of a PPG sensing device for measuring light signals reflected from a subject seated on a toilet, according to an embodiment.

FIG. 2 is a flow chart of an example method of using a PPG sensing device for measuring signals reflected from a subject seated on a toilet, according to an embodiment.

FIG. 3 is a perspective view of a PPG sensing device for measuring light signals reflected from a subject seated on a toilet, according to an embodiment.

FIG. 4 is a partially exploded perspective view of the PPG sensing device for measuring light signals reflected from a subject seated on a toilet depicted in FIG. 3

FIG. 5A is a top view of the PPG sensing device for measuring light signals reflected from a subject seated on a toilet depicted in FIG. 3.

FIG. 5B is a side view of the PPG sensing device for measuring light signals reflected from a subject seated on a toilet depicted in FIG. 3.

FIG. 6A is a perspective view of the PPG sensing device depicted in FIG. 3, displaying a first light source, and a schematic representation of three possible pathways (A1, A2 and A3) in which light can travel from the first light source to three different light sensors and/or detectors included in the PPG sensing device.

FIG. 6B is a perspective view of the PPG sensing device depicted in FIG. 3, displaying a second light source, and a schematic representation of three possible pathways (B1, B2 and B3) in which light can travel from the second light source to three different light sensors and/or detectors included in the PPG sensing device.

FIG. 6C is a perspective view of the PPG sensing device depicted in FIG. 3, displaying a third light source, and a schematic representation of three possible pathways (C1, C2 and C3) in which light can travel from the third light source to three different light detectors included in the PPG sensing device

FIG. 7A is a perspective view of a component of the PPG sensing device depicted in FIG. 3, displaying a light source, and a light sensor and/or detector.

FIG. 7B is a perspective view of a light source included in the PPG sensing device depicted in FIG. 3.

FIG. 8 is a top view of the sensing device depicted in FIG. 3, displaying three light sources and three light sensors and/or detectors included in the sensing device, and the distances that separate each light source to each sensor and/or detector.

FIGS. 9A, 9B, and 9C are a top, side, and bottom view, respectively, of a toilet seat including a set of sensors for monitoring signals (e.g., PPG signals, loads or forces, and ECG signal) associated with various physiological data or conditions of an individual, according to an embodiment.

FIG. 10 schematically depicts a network of devices for monitoring physiological conditions of a subject, according to an embodiment.

FIG. 11 is a flow chart of an example method of dynamically adjusting readings received from light sensors and/detectors of a PPG sensing device, according to embodiments.

FIG. 12 schematically depicts a processing module of a PPG sensing device for dynamically adjusting readings receiving from light sensors and/or detectors of a PPG sensing device, according to embodiments.

### DETAILED DESCRIPTION

The embodiments described herein relate generally to health monitoring systems and devices, and more particularly to systems, devices, and methods for monitoring blood circulation characteristics of an individual seated on an excretion collection device or waste receptacle such as, for example, a toilet. In some embodiments, systems, devices, and methods described herein can measure electromagnetic and/or optical signals reflected from an individual seated on a toilet, which can be used to estimate, determine, and/or monitor volumetric blood flow variations of the individual. The determined volumetric variations of blood flow can then be used to monitor certain physiological data or conditions of the individual and to inform the individual and/or healthcare providers of changes in such data or conditions necessitating certain therapies, treatments, lifestyle changes, etc.

Most individuals use toilets or other types of waste receptacles on a daily basis. Accordingly, health monitoring that can be conducted while an individual is seated on a toilet can provide an unobtrusive way of regularly monitoring information about that individual. Measures such as volumetric blood flow variations of an individual seated on a toilet can be useful for monitoring certain conditions of the individual, such as, for example, cardiac activity and/or cardiac or vascular health of the individual.

Conventional systems, devices, and/or methods for monitoring cardiac activity and/or cardiac or vascular health of an individual typically include electrocardiogram (ECG) devices. ECG devices record the electrical activity of the heart originating from the depolarization of the conductive pathway of the heart and the cardiac muscle tissues during each cardiac cycle. Although ECG devices can provide accurate measurements of cardiac activity of individuals, these devices require the use of multiple electrodes disposed and/or placed at specific body locations. For example, a traditional ECG device may require the use of three electrodes placed on different body locations such as a right arm, a left arm, and a right leg. The need for specific body locations for the ECG electrodes constitute a shortcoming of ECG devices that translates in poor user flexibility, portability, and/or convenience. Alternative devices for monitoring cardiac activity and/or cardiac health which can overcome the shortcomings of ECG devices include sensors such as photoplethysmography (PPG) sensors. PPG sensors can generate and direct a light signal to a region and/or a tissue of an individual, and then measure light reflected and/or transmitted by the region and/or tissue of the individual. The amount of light absorbed, reflected and/or transmitted by the region and/or tissue can be associated with volumetric blood flow variations of the individual, as further described herein. PPG sensors are commonly placed in various anatomic positions, including the earlobes, forehead, or fingertip of individuals. One major difficulty of PPG sensors is their inaccuracy in tracking the PPG signal during routine activities. This limitation stems from the fact that PPG signals are very susceptible to motion artifacts caused by movement of the individual. For example, in some instances, movements of the individual (e.g., movements of the head, arms, and/or hands) or movements caused by skin deformation at the site where the PPG sensor is located can cause sudden changes in the position and/or orientation of the PPG sensor with respect to the tissue and/or region of the individual being illuminated. These changes of position and/or orientation can have an impact on the amount of light reflected by the individual that can be detected by the PPG sensor, resulting in inaccurate readings. The changes of position and/or orientation of the PPG sensor can also alter the path length traveled by the light prior to reaching the sensor and/or detector of the PPG sensor, which can change the amount of light absorbed and/or reflected by the tissue and lead to inaccurate readings. Moreover, alternative factors such as environmental noise and/or temperature can also have an impact on the PPG signal, which consequently affects the accuracy of the monitored cardiac activity and/or health of the individual.

The systems, devices, and methods described herein address the limitations of current PPG devices by providing sensing devices that can measure multiple PPG signals reflected from an individual when the individual is seated on an excretion collection device or waste receptacle such as, for example, a toilet. These PPG signals can significantly reduce and/or eliminate motion artifacts and provide high quality data that can be used to estimate, determine, and/or monitor volumetric blood flow variations of an individual with an improved accuracy and reproducibility. The devices disclosed herein can include one or more light sources configured to generate and/or emit light signals (PPG signals) that travel following different and/or distinct paths directed to the buttocks region of the individual seated on the toilet. The devices can also include one or more sensors and/or detectors disposed at different locations within the sensing devices and configured to detect, sense, and/or measure light signals after being reflected from the individual seated on the toilet. More specifically, each sensor and/or detector can be configured to detect, sense, and/or measure light signals after the light signal has traveled a predetermined and/or predefined path and has been reflected from the individual seated on the toilet. Various sensing or monitoring systems can be used to monitoring cardiac activity and/or cardiac or vascular health based on light signals (e.g., PPG signals). Suitable examples are described in U.S. Pat. No. 10,292,658, titled, “Apparatus, System, And Method For Mechanical Analysis Of Seated Individual,” issued May 21, 2019 (“the '658 patent”), which is incorporated herein by reference.

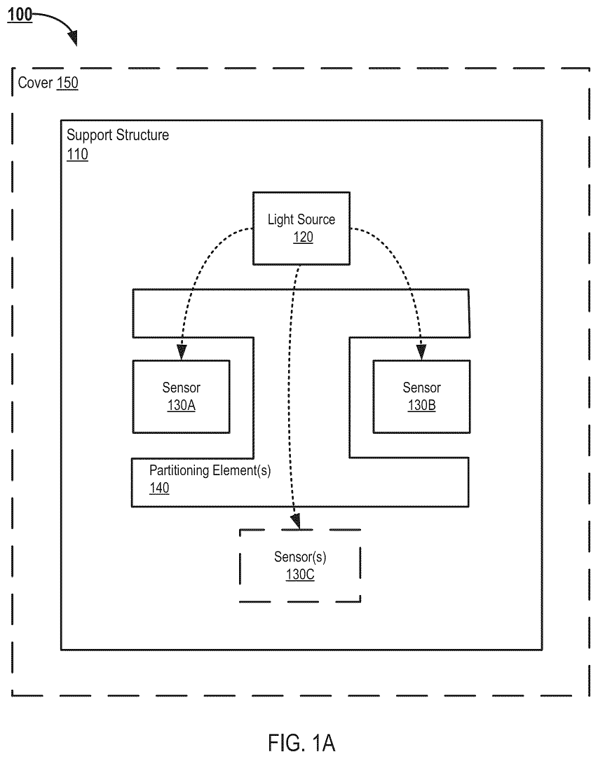

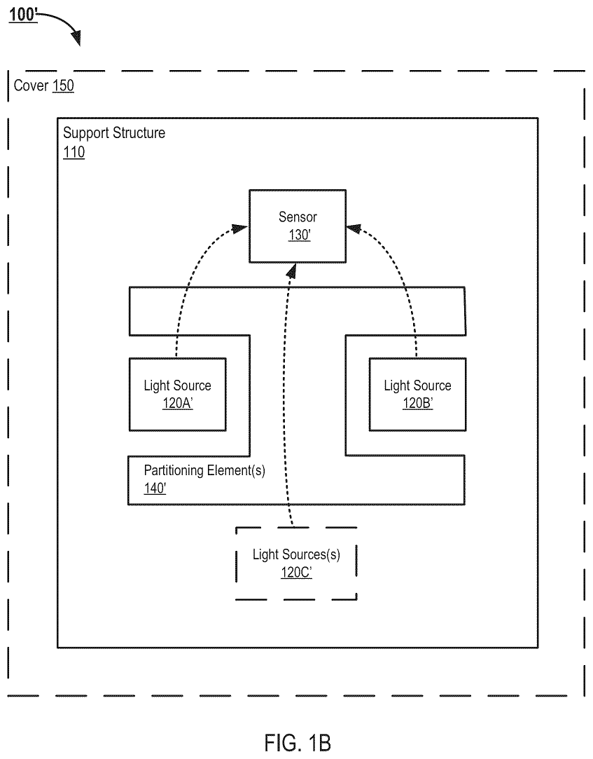

In some embodiments, a PPG sensing device described herein can include a light source and multiple sensors that can measure PPG signals across two separate sensing paths. For example, FIG. 1A shows a schematic illustration of an example PPG sensing device 100 for monitoring physiological data such as PPG signals, according to some embodiments. The PPG sensing device 100, which can also be referred to as the “sensing device 100” or the “device 100,” can include a support structure 110, a light source 120, a first sensor or detector 130A, a second sensor or detector 130B, and one or more partitioning element(s) 140. Optionally, in some embodiments, the sensing device 100 can also include one or more additional sensor(s) or detector(s) 130C and a cover 150.

The support structure 110 can support one or more components of the sensing device 100, such as the light source 120, the first sensor 130A, the second sensor 130B, the optional sensor(s) 130C, and/or the partitioning element(s) 140. In some embodiments, the support structure 110 can include a circuit board or similar structure, e.g., for mounting one or more components of the sensing device 100. In some embodiments, the support structure 110 can be disposed, integrated into and/or directly attached to the toilet. The support structure 110 can be any suitable shape, size, or other configuration. The support structure 110 can be formed of any suitable material having sufficient structural strength and rigidity, including, for example, metal, glass, ceramic, and/or polymers. In some embodiments, the support structure can be formed of conductive and insulating layers, e.g., for allowing the assembly of electrical or data components. In some embodiments, the support structure 110 can include multiple portions that can be coupled and/or assembled together, e.g., to form and/or define a surface for receiving the components of the sensing device 100. That is, in some implementations, the support structure 110 can be modular. Alternatively, in other embodiments, the support structure 110 can be made of a monolithic structure. The light source 120 can be a component disposed on the support structure 110 and configured to generate and/or emit light of predetermined characteristics. The sensor 130A, the sensor 130B, and/or the optional sensor(s) 130C (collectively, the sensor(s) 130) can be configured to detect, sense, and/or measure light generated by the light source 120 that is reflected by the individual seated on the toilet.

In some embodiments, the PPG sensing device 100 can include one or more spacers disposed and/or coupled to a surface of the support structure 110. The spacers can be made of a rigid material sized and dimensioned to collectively set and/or define a fixed (e.g., constant) predetermined distance and/or gap between a cover 150 and one or more components of the PPG sensing device 100 disposed on the support structure 110 (e.g., a light source 120, a sensor 130, and/or a partitioning element 140) when the cover 150 is attached and/or coupled to the PPG sensing device 100. Said in other words, the spacers can be disposed and/or coupled to a surface of the support structure 110 to receive the cover 150 and generate a three-dimensional space (e.g., an interior volume) between the surface of the support structure 110 and the cover 150. This interior volume can accommodate components of the PPG sensing device 100 such that a top surface of each of the components, (e.g., a light source 120 and/or a sensor 130) is disposed at a fixed (e.g., constant) predetermined distance from the cover 150, with the fixed predetermined distances being defined by the height of the spacers. For example, in some embodiments the PPG sensing device 100 may include a support structure 110, a light source 120, a sensor 130, a partitioning element 140, and a plurality of spacers having a predetermined height. The plurality of spacers can be disposed on the PPG sensing device 100 (e.g., coupled to the support structure 110) and configured to collectively set and/or define a first fixed predetermined distance and/or height between a top surface of the light source 120 and the cover 150, and a second predetermined fixed distance and/or height between the sensor 130 and the cover 150.

The spacers can prevent that loads and/or forces applied to the cover 150 when, for example, a subject is seated on the toilet and contacting the PPG sensing device 100, deform the cover and alter and/or reduce the distances between components of the PPG sensing device (e.g., the light source(s) 120 and/or the sensor(s) 130) and the cover 150. In some embodiments in which the PPG sensing device 100 includes a partitioning element 140 made of and/or comprising a flexible and/or a compressible material, the PPG sensing device 100 may preferably include one or more spacers coupled to the support structure 110 and/or to the cover 150. In such embodiments, forces and/or loads applied to the cover 150 when a subject is seated on the toilet contacting the PPG sensing device 100 can deform and/or move the cover 150 compressing the partitioning element 140. In the absence of one or more spacers, the cover 150 could be deformed and/or moved by the loads and forces applied when the subject is seated on the toilet contacting the PPG sensing device and reduce and/or alter the distances between the cover 150 and each of the light sources 120 and/or sensors 130. Furthermore, in some extreme cases, the cover 150 may become sufficiently deformed due to the loads and/or forces such that a portion of the cover 150 may physically contact at least a light source 120 and/or at a sensor 130 of the PPG sensing device 100. Consequently, the use of spacers disposed on and/or coupled to the support structure 110 and/or the cover 150 prevents altering the distances between component of the PPG sensing device 100 such as light sources 120 and/or sensors 130 and the cover 150.

The spacers can have any size and/or shape. In some embodiments the spacers can have a shape defined by a predetermined height and a suitable cross sectional area including, but not limited to triangular, circular, square, hexagonal, and/or a polygon. For example, in some embodiments the spacers can be cylindrical posts and/or columns disposed about the support structure. In other embodiments, the spacers can be cuboid structures characterized by a length, a width, and a height (e.g., the predetermined fixed height of the spacers) and disposed about the support structure. In some embodiments, the spacers can be disposed around the perimeter of the support structure 110. In some embodiments, the spacers can be disposed on an interior portion of the support structure 110 around other components of the PPG sensing device 100. The spacers can be made of a rigid and/or stiff material which can be deformed under loads and/or forces associated to the weight of a subject and/or individual. For example, in some embodiments the spacers can be made of metals and metal alloys including, but not limited to, aluminum, steel, stainless steel, nickel, and/or copper. In some embodiments the spacers can be made of rigid polymeric materials and/or plastics including acrylic, epoxy, polyimide, polystyrene, polyethylene terephthalate (PET) and the like. As described above, in some embodiments the spacers can be disposed and/or coupled to the support structure 110. Alternatively and/or optionally, in some embodiments the spacers can be disposed and/or coupled to the cover 150. In some embodiments, the spacers can include a first portion disposed and/or coupled to the support structure 110, and a second portion disposed and/or coupled to the cover 150.

In some embodiments, the PPG sensing device 100 can be integrated into or coupled to a seat of a toilet (or other waste receptacle) and be configured to measure PPG signals of an individual seated on the toilet (or other waste receptacle). The PPG sensing device 100 can be disposed on the top surface of the toilet seat such that the PPG sensing device 100 can emit and capture reflected light from an individual when that individual is seated on the toilet seat. In use, the light source 120 can be activated to generate light, which can be directed toward tissue of the individual seated on the toilet. The light can penetrate through the tissue and blood vessels, and a certain amount of light can reflect back toward the PPG sensing device 100. The reflected light can be analyzed to derive physiological information about the individual, e.g., PPG waveform, heart rate, cardiac cycle, respiration, etc.

The partitioning element(s) 140 can be one or more suitable components disposed on the support structure 110 surrounding the light source 120 and the sensors 130. The partitioning element(s) 140 can function as light-blocking structures that are configured to prevent light from the light source 120 from saturating the sensors 130. In some embodiments, the partitioning element(s) 140 can be configured to direct light toward a target tissue of a subject, or a desired sensor 120 (e.g., after being reflected by the target tissue). In some embodiments, the partitioning element(s) 140 can prevent the light from propagating in an unwanted direction (e.g., escaping and/or leaking from the sensing device 100 through the cover 150 prior to reaching a target tissue and after being reflected from the target tissue), and instead restrict the direction of propagation of the light towards a desired direction and/or target. For example, the partitioning element(s) 140 can be configured to direct light generated by the light source 120 toward the tissue of an individual, such that the generated light penetrates through the tissue before being reflected back and being captured by a sensor 130. The partitioning element(s) 140 can shield the light sensors 130 from light generated by the light source 120 and other environmental light noise, while allowing light that is reflected from the individual's tissue to be captured. In other words, the partitioning element(s) 140 can be configured to reduce lateral leakage of light (e.g., electromagnetic radiation) from the light source 120 to the sensor 130 such that the light and/or electromagnetic radiation measured by the sensor 130 is substantially light and/or electromagnetic radiation generated by the light source 120 and reflected by the tissue of a subject, when the subject is seated on the toilet. In some embodiments, one or more sensor(s) 130 may be disposed in the PPG sensing device 100 to measure a portion of the electromagnetic radiation generated by one or more light source(s) 120 of the PPG sensing device 100, wherein the portion of the electromagnetic radiation corresponds to electromagnetic radiation reflected by tissue of a subject, when the subject is seated on the toilet. In such embodiments, the partitioning element(s) 140 may be disposed about the one or more light source(s) 120 and the one or more sensor(s) 130 to prevent electromagnetic radiation other than the portion of electromagnetic radiation from being detected by the one or more sensor(s) 130. In operation, the light source 120 can generate light which can be directed by the partitioning element(s) 140 toward a seated individual's tissue. The light so guided can penetrate through an incident surface on or near the buttocks region of the individual seated on the toilet. Then, after being reflected from the tissue of the individual seated on the toilet, the reflected light can be detected by a sensor 130.

In some embodiments, the portioning element(s) 140 can include one or more gaskets disposed around the light source 120 and/or the sensor(s) 130, e.g., to isolate the light source 120 and the sensor(s) 130. In some embodiments, the partitioning element(s) 140 can include multiple portions that can be coupled and/or assembled together. That is, in some embodiments, the partitioning element(s) 140 can be modular. Alternatively, in other embodiments, the partitioning element(s) 140 can be made of a monolithic structure. The partitioning element(s) 140 can be made of any suitable material. For example, in some embodiments the partitioning element(s) 140 can be made of flexible, elastic materials including ethylene vinyl acetate (EVA), polyethylene, polyurethane, rubber, etc., e.g., to facilitate disposing and/or placing the partitioning element(s) 140 tightly between the support component 110 and the optional cover 150. In some embodiments, the partitioning element(s) 140 can be made of polymeric materials having sufficient mechanical properties and ease of processability such that the partitioning element(s) 140 can be processed in different shapes, sizes with high tolerances. For example, in some embodiments, the partitioning element(s) 140 can be made of polymeric materials such as nylons, polyesters, polycarbonates, polyacrylates, polysiloxanes (silicones), polymers of ethylene-vinyl acetates and other acyl substituted cellulose acetates, non-degradable polyurethanes, polystyrenes, polyvinyl chloride, polyvinyl fluoride, poly (vinyl imidazole), chlorosulphonate polyolefins, polyethylene oxide, and/or blends and copolymers thereof.

In some embodiments, the sensing device 100 can optionally include a cover 150. The cover 150 can be made of a material that is transparent such that the light generated by the sensing device 100 can pass through the cover 150 and into a seated individual's tissue. In some embodiments, the cover 150 can be coupled to the support structure 110 and/or partitioning element(s) 140 and form an enclosure that contains or houses the light source 120 and the light sensor(s) 130 of the PPG sensing device 100. In some embodiments, the cover 150 can form a flat or smooth surface on which a seated individual's tissue can contact. In some embodiments, the cover 150 can form a curved surface on which a seated individual's tissue can contact. In some embodiments, the top or exposed surface of the cover 150 can be flush with a surface of a toilet seat. The partitioning element(s) 140 can be designed to extend to the cover, thereby guiding light from the light source 120 toward the cover 150 and then into tissue in contact with the cover, and light reflected from the tissue can pass through the cover 150 to one or more of the sensors 130. In some embodiments, the cover 150 can include multiple portions that can be coupled and/or assembled together, e.g., to form the enclosure that contains or houses the light source 120 and the light sensor(s) 130 of the sensing device 100. That is, in some embodiments, the cover 150 can be modular. Alternatively, in other embodiments, the cover 150 can be made of a monolithic structure.

The optional cover 150 can be configured to protect the various components of the sensing device 100 disposed below the cover 150. In some embodiments, the cover 150 can be made of one or more materials that do not absorb visible and/or IR light generated by the light source 120. For example, in some embodiments the cover 150 can be made of materials that absorb negligible amounts of IR light such as for example, quartz, Germanium (Ge), Zinc Selenide (ZnSe), sapphire phase alumina (Al₂O₃), calcium fluoride (CaF₂), potassium bromide (KBr), Barium Fluoride (BaF₂), Silicon (Si), gallium Arsenide (GeAs) and the like. In some embodiments, the cover 150 can be made of one or more materials that do not absorb visible light (e.g., red, green, violet, orange, etc.) generated by the light source 150. For example, in some embodiments the cover 150 can be made of materials that absorb negligible amounts of visible light such as optical glass and/or pyrex, quartz, sapphire, magnesium fluoride (MgF₂), Lithium fluoride (LiF₂), fused silica, and/or polymeric materials such as poly methyl methacrylate (PMMA), polyurethane, polycarbonate, polystyrene, and the like. In some embodiments, the cover 150 can be a multilayer material that comprising multiple layers and/or coatings stacked on top of each other, with each layer having one or more different optical properties. For example, in some embodiments, the cover 150 can be made of diamond-like carbon coated with Germanium. In some embodiments, the cover 150 can be made of sapphire alumina coated with a sodium chloride (NaCl) layer.

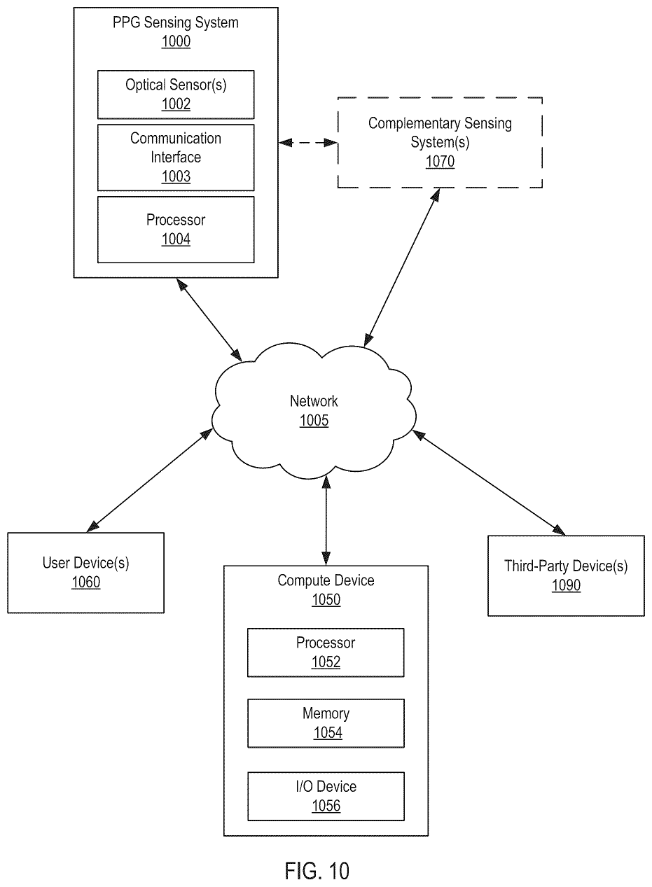

In some embodiments, the sensing device 100 can include and/or be operatively coupled to a processor (e.g., an onboard processor and/or a processor of a separate compute device (see FIG. 10)) configured to activate and/or control the operation of one or more components of the sensing device 100. For example, the processor can be configured to send electrical signals to the light source 120 to activate or deactivate the light source 120. In some embodiments, the processor can also send signals to the light source 120 to control or set one or more properties of the light being generated by the light source 120. For example, the processor can control the light source 120 to generate light having a specific frequency or intensity. In some embodiments, the processor can be configured to receive signals from the sensors 130 indicative of the light reflected from the individual in response to the light directed to the individual by the light source 120. The reflected light measured by the sensor(s) and/or detector(s) 130 can be received and analyzed by the processor to estimate, determine and/or monitor various physiological data or conditions of the individual seated on the toilet, as further described herein.

The light source 120 can be any suitable illumination component and/or device configured to emit electromagnetic radiation having one or more predetermined characteristics. In some embodiments the light source 120, which can also be referred to herein as the illumination source 120, can include a light-emitting diode (LED), a Xenon Energy discharge lamp (XED), a fluorescent light source and or lamp, a mercury light source, an incandescent light source, or the like. In some embodiments, the light source 120 can be configured to generate electromagnetic radiation having one or more predetermined characteristics, such as, for example, a predetermined frequency or range thereof. For example, the light source 120 can be configured to generate visible light. Alternatively, or additionally, the light source 120 can be configured to generate infrared (IR) light. Alternatively, or additionally, the light source 120 can be configured to generate a range of different types of light (e.g., visible light, UV-vis light, and/or IR light).

In some embodiments, the light source 120 can be coupled to one or more filters and/or lenses designed to change, manipulate and/or precondition the electromagnetic radiation (e.g., light) generated by the light source 120. For example, in some embodiments, one or more filters can be used to selectively transmit light having certain frequencies from the light source 120. In some embodiments, the light source 120 (with or without a filter) can be configured to generate light having a wavelength of about 500 nanometers (nm) to about 565 nm (which corresponds to green light), including all values and sub-ranges therebetween. In some embodiments, the light source 120 (collectively with or without a filter) can be configured to generate light having a wavelength of about 625 nm to about 740 nm (which corresponds to red light), including all values and sub-ranges therebetween. In some embodiments, the light source (with or without a filter) can be configured to generate light having a wavelength of about 750 nm to about 1 mm (which corresponds to IR light), including all values and sub-ranges therebetween. In some embodiments, the light source 120 (with or without a filter) can be configured to generate light having a wavelength of between about 400 nm to about 1 mm, including all values and sub-ranges therebetween.

In some embodiments, the light source 120 can optionally include one or more lenses and/or light guides coupled to the light source 120. The lenses and/or light guides can be configured to facilitate transporting and/or directing the electromagnetic radiation (e.g., light) generated by the light source 120 towards a specific target region, portion and/or surface of the individual seated on the toilet. For example, in some embodiments, the light source 120 can include a LED coupled to a light guide. The light guide can receive light emitted by the LED and guide the received light to a target point and/or region adjacent to the buttocks of the individual seated on the toilet. The light guide can be an air column or be formed of a suitable optical grade material such as, for example, acrylic resin, polycarbonate, epoxy resins, and/or glass. In some embodiments, one or more lenses and/or light guides can be disposed on the sensing device 100 such that light reflected from the individual seated on the toilet can be collected, focused, or transmitted to the sensors 130 via the one or more lenses and/or light guides.

The sensors 130 shown in FIG. 1A (e.g., sensor 130A, the sensor 130B, and/or the one or more optional sensor(s) 130C) can be and/or include any suitable photodetector and/or photosensor configured to sense, detect, and/or measure light signals or readings (e.g., PPG signals). In some embodiments, the sensors 130 can be configured to sense, detect, and/or measure light signals reflected from the individual seated on the toilet having a wavelength of at least about 625 nm and no more than about 740 nm (which correspond to red light), including all values and sub-ranges therebetween. In some embodiments, the sensors 130 can be configured to sense, detect, and/or measure light signals reflected from the individual seated on the toilet having a wavelength of at least about 500 nm and no more than about 565 nm (which correspond to green light). In some embodiments, the sensors 130 can be configured to sense, detect, and/or measure light signals reflected from the individual seated on the toilet having a wavelength of at least about 750 nm and no more than about 1 mm (which correspond to IR light). In some embodiments, the sensors 130 can be configured to measure light in a broad range of wavelengths, e.g., from about 400 nm to about 1 mm, including all values and sub-ranges therebetween.

In some embodiments, the sensor(s) 130 can include photodiodes. The photodiodes can be made of a silicon semiconductor material comprising P-N junctions that can absorb incident photons from a light beam and generate an electrical current response signal proportional to the number of photons absorbed. In use, the magnitude of the current generated by the photodiode can be correlated to the amount and/or intensity of the light signals reflected from the individual seated on the toilet. In some embodiments, the sensor(s) and/or detector(s) 130 can include one or more photodiodes configured to detect and/or measure light signals reflected from the individual seated on the toilet, with the reflected light signals having multiple wavelengths, for example wavelengths of at least about 520 nm and no more than about 565 nm (which correspond to green light), at least about 625 nm and no more than about 750 nm (which correspond to red light), and/or at least about 750 nm and no more than about 1 mm (which correspond to IR light).

In some embodiments, the sensor(s) 130 can include and/or be photoresistors (e.g., a light-dependent resistor or LDR). The photoresistors can be made of a material that can decrease its electrical resistance proportional to the amount of light signals received. The photoresistors can be coupled and/or integrated to an electrical circuit which is used to measure the changes in the electrical resistance of the photoresistors as light signals reflected from the individual seated in the toilet is received by the photoresistors. In use, the absolute and/or relative magnitudes of the electrical resistance of the photoresistors can be correlated to the amount and/or intensity of the light signals reflected from the individual seated on the toilet. In some embodiments, the sensor(s) 130 can include one or more photoresistors configured to receive light signals reflected from the individual seated on the toilet, with the reflected light having multiple wavelengths, for example wavelengths of at least about 520 nm and no more than about 565 nm (which correspond to green light), at least about 625 nm and no more than about 750 nm (which correspond to red light), and/or at least about 750 nm and no more than about 1 mm (which correspond to IR light).

In some embodiments, the sensor(s) 130 can include and/or be photovoltaic cells. The photovoltaic cells can include single-crystal silicon P-N junctions that can generate a voltage and/or electrical potential difference proportional to the intensity of light signals received by the photovoltaic cell. In use, the magnitude of the potential generated by the photovoltaic cells can be correlated to the amount and/or intensity of the light signals reflected from the individual seated on the toilet. In some embodiments, the sensor(s) 130 can include one or more photovoltaic cells configured to receive light signals reflected from the individual seated on the toilet, with the reflected light signals having multiple wavelengths, for example wavelengths of at least about 520 nm and no more than about 565 nm (which correspond to green light), at least about 625 nm and no more than about 750 nm (which correspond to red light), and/or at least about 750 nm and no more than about 1 mm (which correspond to IR light).

The sensor(s) and/or detector(s) 130 can be coupled to a processor (e.g., an onboard processor and/or a processor of a separate compute device (see FIG. 10)) that can use the information collected by the sensor(s) and/or detector(s) 130 to evaluate various physiological data or conditions of the individual seated on the toilet. In some embodiments, data collected by the sensor(s) and/or detector(s) 130 (e.g., PPG data) can be combined with data produced by other sensor(s), such as, for example, an electrocardiogram (ECG) sensor, a ballistocardiogram (BCG) sensor, a temperature sensor, force and/or weight sensors, etc. to estimate relevant information for the medical analysis of cardiac and vascular function. With the information from the sensor(s) 130 and/or additional sensors, the processor can determine or monitor one or more of the following physiological parameters about an individual or subject: heart rate, heart rate variability, left ventricular ejection time, pre-ejection period, flow velocity, pulse transit time (e.g., based on PPG, ECG or BCG data), blood pressure, cardiac output, cardiac contractility, abnormal heart function, blood oxygenation levels (e.g., SpO₂), respiration rate, stress levels (e.g., via heart rate variability), body weight, cardiac waveform characteristics (e.g., magnitudes and/or intervals), etc. Suitable examples of processing and/or evaluation of sensor data are described in the '658 patent, as incorporated by reference above.

In some embodiments, a PPG sensing device described herein can include a sensor and multiple light sources that can measure PPG signals across at least two sensing paths. For example, FIG. 1B shows a schematic illustration of another an example sensing device 100′ for monitoring physiological data such as PPG signals, according to some embodiments. The sensing device 100′ can include components that are structurally and/or functionally similar to the sensing device 100, and therefore certain details of these components are not described herein again. For example, the device 100′ can include a support structure 110, a first light source 120A′, a second light source 120B′, an sensor and/or detector 130′, and one or more partitioning element(s) 140′. Optionally, in some embodiments the sensing device 100′ can include one or more additional light source(s) 120C′ and/or a cover 150.

The support structure 110 can support the components of the sensing device 100′, such as, for example, the first light source 120A′, the second light source 120B′, the one or more optional light source(s) 120C′, the sensor 130′, and/or the partitioning element(s) 140′. The first light source 120A′, the second light source 120B′, the one or more optional light source(s) 120C′ (collectively, the light source(s) 120′) can be disposed on the support structure 110 and configured to generate light of predetermined characteristics, similar to the light source 120 described above with reference to the sensing device 100. The sensor 130′ can be any suitable sensor and/or detector configured to detect, sense, and/or measure light generated by the light source(s) 120′, after the light has been reflected by the individual seated on the toilet. The partitioning element(s) 140′ can be configured to shield the sensor 130′ from light generated by the light source(s) 120′ and other environmental light noise, while allowing light that is reflected from the individual's tissue to be captured. In other words, the partitioning element(s) 140′ can be configured as light-blocking structures that block light generated by the light source(s) 120′ from the sensor 130′.

The PPG sensing devices 100 and 100′ can be configured to capture PPG signals or other physiological data across multiple sensing paths. For example, each light source 120, 120′ can be paired with each sensor 130, 130′ to capture PPG signals across multiple sensing paths. The sensing device 100, as shown in FIG. 1A, can have at least two sensing paths, e.g., a first sensing path between light source 120 and sensor 130A, and a second sensing path between light source 120 and sensor 130B. The sensing device 100′, as shown in FIG. 1B, can have at least two sensing paths, e.g., a first sensing path between light source 120A′ and sensor 130′, and a second sensing path between light source 120B′ and sensor 130′. Each of sensing device 100 and 100′ can also have additional sensing paths, e.g., between additional light sources and/or sensors. The sensing paths of PPG sensing device 100 and 100′ can each be different, e.g., have a different path length, be located in a different location, be associated with a different type or wavelength of light (e.g., red, IR, green), be associated with a different intensity of light, etc. In use, light generated by the light sources 120, 120′ can travel through their independent predetermined paths, penetrate through tissue of an individual seated on a toilet or other waste receptacle, and then after being reflected by the tissue, continue to a sensor 130, 130′.

The PPG sensing devices 100 and 100′, by being configured to generate at least two paths for measuring PPG signals, can have certain advantages. By having multiple paths for measuring PPG signals, the PPG sensing device 100 and 100′ can avoid inaccurate measurements (or biased measurements) associated with differences in tissue, differences in sitting pressure and/or location, and/or differences in skin tone, weight, and/or other physical characteristics of a seated individual. For example, when a seated individual has tissue scarring in one region, certain pathways may lead to no measurement signal or weak measurement signals due to the scarred tissue preventing or reducing light penetration. Differences in sitting pressure and/or location can also affect measurements (e.g., where individuals sit by applying pressure differently and/or have different posture). In some cases, longer path lengths may be less suitable for certain individuals, e.g., where skin tone or thickness of tissue may prevent light from being detected by a sensor. But longer path lengths also lead to deeper light penetration, and therefore may also be useful for capturing signals that are deeper in tissue. With all of these factors, it can be beneficial to have PPG sensing devices that can measure signals over multiple paths, with some different paths having different lengths and/or being in different locations. The signals from each of these sensing paths can then be aggregated or evaluated together, e.g., to overcome issues with loss of signals or weak signals and/or to ensure that the captured data is not biased due to environmental factors.

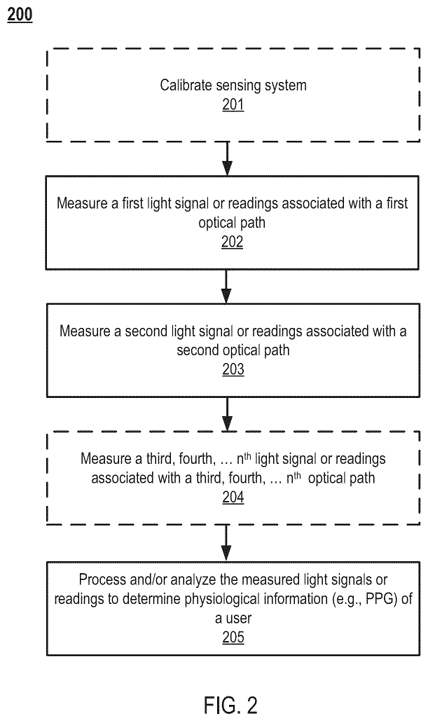

FIG. 2 shows an example method 200 of using the systems and devices described herein (e.g., sensing device 100, 100′, 300, etc.) when attached, installed, or integrated onto a toilet or other waste receptable, according to embodiments. The sensing system can optionally be calibrated, at 201. In some embodiments, calibration can be performed by capturing data using one or more sensor(s) (e.g., sensors 130, 130′) of the sensing device under predefined conditions, and then sending that data to a processor (e.g., an onboard processor and/or processor associated with an external compute device such as for example, the user device 1060, and/or the compute device 1050 shown in FIG. 10) to have the processor calibrate the sensing device. In some instances, the system can be calibrated by first collecting data while a user is not seated on the toilet, and then collecting data while the user is seated on the toilet. In some embodiments, the system can be calibrated during manufacturing. In some embodiments, the system can be calibrated during or after installation of the system, e.g., to account for different attachments of the system components to a toilet seat (or other seat) and/or differences across users. For example, with the PPG sensing paths described above, during a calibration phase, a processor can determine whether certain sensing paths produce weak, saturated, or inaccurate data. In some embodiments, the processor can then vary one or more parameters of the light source(s) and/or sensor(s) to account for weak or inaccurate data, e.g., increase an intensity of light being generated by a light source, reduce an intensity of light being generated by a light source, change a frequency of light being generated by a light source (e.g., change to using green light instead of red/IR light), or change any other suitable operational parameters.

The sensing system can be used to capture physiological data (e.g., PPG signals) of an individual or subject seated on a toilet. In particular, the sensing system can be configured to measure light signals across multiple sensing paths, e.g., as described above with respect to PPG sensing devices 100, 100′. For example, a first light signal or reading associated with a first path can be measured by one of the sensor(s) of the sensing system, at 202. The first path can start at a light source (e.g., 120, 120′), which generates light that is directed at a portion of the tissue of the seated individual. The light that is then reflected from the individual is received and measured by an sensor of the sensing system, completing the first path.

At 203 and 204, additional light signal(s) or reading(s) (e.g., a second, third, fourth, . . . and nth light signal or reading associated with a second, third, fourth . . . and nth pathway, respectively) can be measured by different light source and sensor pairs of the sensing system. For example, light can be generated by a second light source (e.g., 120, 120′), and reflected light can be captured by a second sensor, following an associated second path. In some embodiments, the second path can be different from the first path such that different information can be captured via the first and second paths. For example, the second path can be substantially different (e.g., have a different direction, trajectory, and/or length) from the first path. In some embodiments, the second path can be substantially similar and/or the same as the first path, but one or more characteristics of the light used in the paths may be different (e.g., red/IR light vs. green light, or higher or lower intensities of light). In some instances, each of the first, second, third, fourth . . . and nth paths associated with the first, second, third, fourth . . . and nth light signals or readings can be different from each other (e.g., each pathway can have a unique direction, trajectory, and/or length). In other instances, some of the paths associated with some of the light signals or readings can be substantially similar and/or the same, while other paths associated with other light signals can be substantially different.

In some instances, a first, second, third, fourth, . . . nth light signal or reading can be measured sequentially. That is, only one light signal or reading is measured at one time, with subsequent light signals or readings being measured at later times, e.g., according to a predefined sequence. For example, a first light signal or reading can be measured by a first sensor during a first time period, and subsequently, a second light signal or reading can be measured by a second sensor (or the same sensor) during a second time period. In other instances, one or more of a first, second, third, fourth, . . . nth light signals or readings can be measured concurrently. That is, first, second, third, fourth, . . . nth light signals or readings can be measured simultaneously by first, second, third, fourth, . . . nth sensors 130. For example, a first and a second light signal or reading can be measured simultaneously by one or more sensors and/or detectors. In such embodiments, light signals in separate paths may or may not interfere with one another, but the signals collectively captured by the light sensor(s) can be processed together to isolate an individual's PPG signal or other physiological data.

At 205, the light signals or readings can be received from the sensors and processed and/or analyzed by a processor (e.g., an onboard processor and/or processor associated with an external compute device such as, for example, the user device 1060, and/or the compute device 1050 shown in FIG. 10). Based on the signals received at the processor, the processor can monitor one or more physiological condition(s) associated with the user. Optionally, the processor can present information of the monitored data to a user and/or provide feedback to a user based on the monitored data, such as through one or more compute devices (e.g., user device 1060, compute device 1050, and/or third-party device 1090 in FIG. 10).

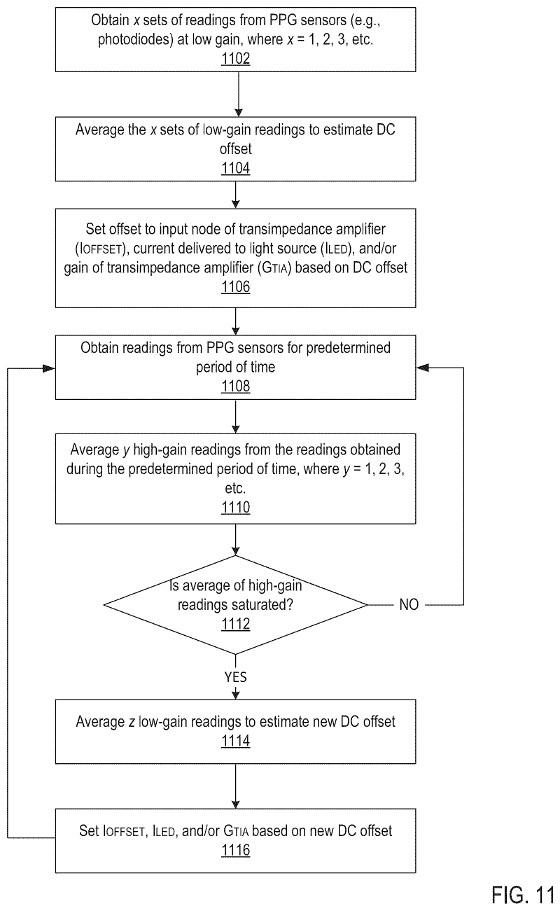

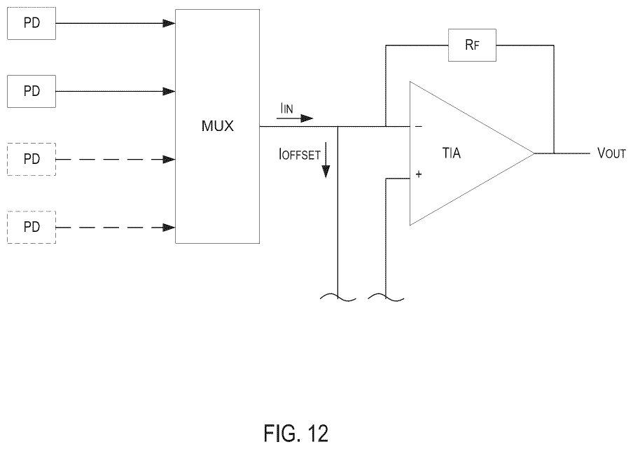

While not specifically depicted in FIG. 2, in some embodiments, the method 200 can also include performing a DC offset, as described below with reference to FIG. 11. The DC offset can be performed in conjunction with measuring the light signals at 202, 203, 204 and processing and/or analyzing the measured data, at 205. The DC offset can be specific to various hardware implementations of systems and devices described herein, including, for example, the hardware configuration depicted in FIG. 12. Further details of the DC offset are described with reference to FIGS. 11 and 12.

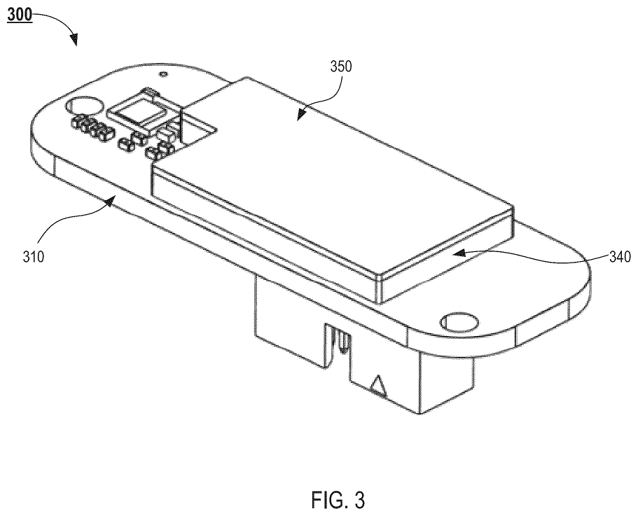

FIGS. 3-7 show different views of a PPG sensing device 300 for monitoring light signals (e.g., PPG signals) reflected from an individual seated on a toilet (e.g., a lavatory), according to some embodiments. The PPG sensing device 300, which can also be referred to herein as the “sensing device 300” or the “device 300,” can be the same or similar in structure and/or function to the sensing devices 100 and 100′ described above with reference to FIGS. 1A-1B. As such, portions and/or aspects of the sensing device 300 can be similar to and/or substantially the same as portions and/or aspects of the sensing devices 100 and 100′ described above with reference to FIGS. 1A-1B, and therefore are not described in detail herein. The sensing device 300 can include a support structure 310, a first light source 320A, a second light source 320B, a third light source 320C, a first sensor 330A, a second sensor 330B, a third sensor 330C, a partitioning element 340, and a cover 350.

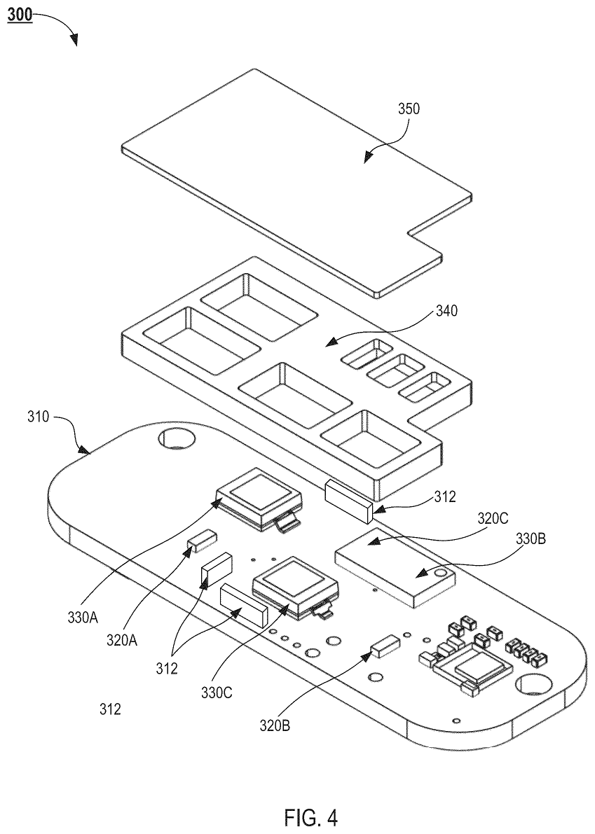

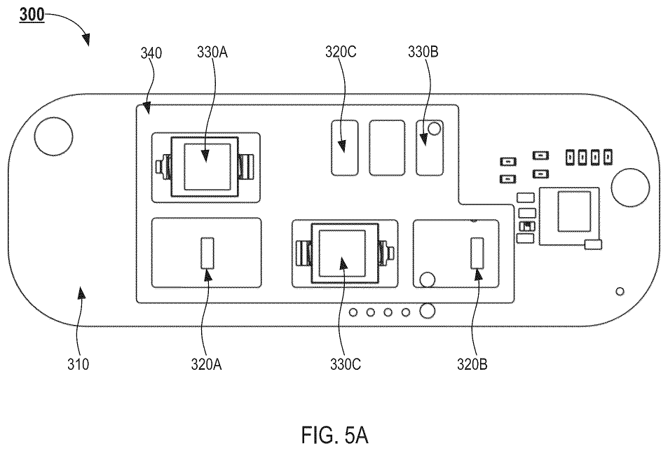

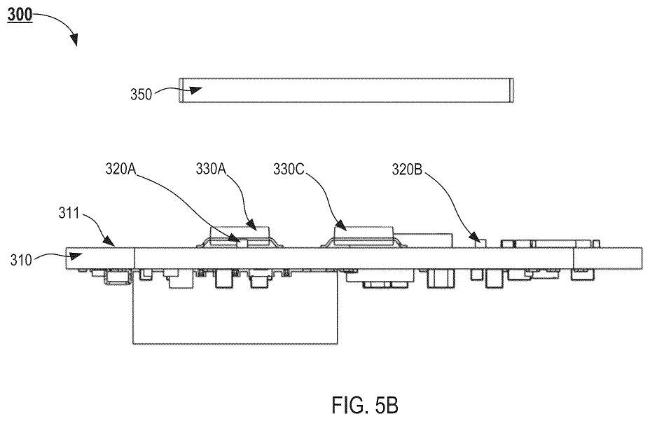

The support structure 310 can support or house various components of the sensing device 300. As shown in FIG. 4, the support structure 310 provides a surface that accommodates the first light source 320A, the second light source 320B, and the third light source 320C, which can be collectively referred to herein as the light sources 320. The support structure 310 can also provide a surface that accommodates the first sensor 330A, the second sensor 330B, and the third sensor 330C, which can be referred to as the sensors 320. The support structure 310 can also support the partitioning element 340 and/or the cover 350. In some embodiments, the support structure can have a rectangular shape, as shown in FIGS. 4-5B. Alternatively, the support structure can have any suitable shape for supporting various components of the PPG sensing device 300. In some embodiments, the corners of the support structure 310 can be rounded, as shown in FIGS. 4-6C. In other embodiments, the corners of the support structure 310 can be sharp or have any suitable shape. FIG. 4 shows in some embodiments the support structure 310 can include one or more spacers 312 disposed about a surface of the support structure 310. The spacers 312 of the PPG sensing device 300 can be similar to and/or the same as the spacers described above with reference to the PPG sensing device 100. For example, the spacers 312 can be made of a rigid material sized and dimensioned to set and/or define fixed predetermined distances and/or gaps between the cover 350 and the components of the PPG sensing device 300 disposed on the support structure 310 (e.g., the light sources 320A, 320B, and 320C, and the sensors 330A, 330B, and 330C) when the cover 150 is attached and/or coupled to the PPG sensing device 300. FIG. 4 shows the spacers 312 are cuboid structures characterized by a length, a width, and a height (e.g., the predetermined fixed height of the spacers). The spacers 312 can be disposed near to and/or around the perimeter of the support structure 310. Additionally and/or optionally, in some embodiments one or more spacers 312 can be disposed on an interior region of the support structure 310 about the light sources 320 and/or the sensors 330 (e.g., an area and/or portion of the support structure surrounded by the perimeter of the support structure 310 on which the light sources 320 and the sensor 330 are disposed).

The light sources 320 (e.g., light source 320A, 320B, and/or 320C) can be any suitable illumination component configured to emit electromagnetic radiation having one or more predetermined characteristics (e.g., wavelength(s) and/or intensity). In some embodiments a light source 320, which can also be referred to herein as an illumination source 320, can include multiple light-emitting diodes (LEDs) that emit electromagnetic radiation of predetermined wavelength(s). For example, in some embodiments, a light source 320 can include a first LED and a second LED. The first LED can be configured to generate and/or emit electromagnetic radiation of a first frequency and/or wavelength (or a range of frequencies and/or wavelengths) that corresponds to the red-light portion of the electromagnetic spectrum. The second LED can be configured to generate and/or emit electromagnetic radiation of a second frequency and/or wavelength (or a range of frequencies and/or wavelengths) that corresponds to the IR portion of the electromagnetic spectrum. In some embodiments, a light source 320 can include a first LED, a second LED, and a third LED. The first LED can be configured to generate and/or emit electromagnetic radiation of a first frequency and/or wavelength (or a range of frequencies and/or wavelengths) that corresponds to the red-light portion of the electromagnetic spectrum, the second LED light can be configured to generate and/or emit electromagnetic radiation of a second frequency and/or wavelength (or a range of frequencies and/or wavelengths) that corresponds to the IR portion of the electromagnetic spectrum, and the third LED light can be configured to generate and/or emit electromagnetic radiation of a third frequency and/or wavelength (or a range of frequencies and/or wavelengths) that corresponds to the green-light portion of the electromagnetic spectrum. In some embodiments, each of the light sources 320A, 320B, and 320C can be substantially the same. Alternatively, one or more of the light sources 320A, 320B, and/or 320C can be different from one another, e.g., emit different wavelength(s) and/or intensities of electromagnetic radiation (e.g., light), include different types of light-generating elements (e.g., LEDs, fluorescent lights, light guides, etc.), include different arrangements and/or number of light-generating elements, etc. In some embodiments, a light source 320 can be disposed as part of a multi-chip package or module, which can be coupled to the support structure 310.

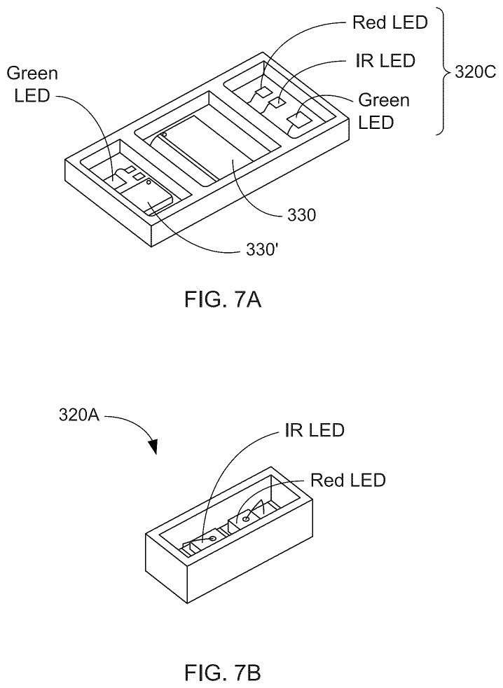

FIGS. 7A and 7B provide detailed views of example light sources. FIG. 7A shows a multi-chip package including a red LED configured to generate and/or emit red light, an IR LED configured to generate and/or emit IR light, and a green LEDs configured to generate and/or emit green light. FIG. 7B shows a multi-chip package including a red LED configured to generate and/or emit red light, and an IR LED configured to generate and/or emit IR light. In the multi-chip package depicted in FIG. 7A, the light source can be included with other electronic components, including, for example, a sensor (e.g., sensor(s) 330, 330′). While the specific arrangements of light and sensor components are depicted in FIGS. 7A and 7B, it can be appreciated that the PPG sensing devices described herein can incorporate any suitable arrangement of light and sensor components.

The light sources 320 can be disposed at different positions, locations, and/or orientations on the support structure 310 such that they can generate light signals that can travel via different paths to one or more sensors, as further described herein. For example, as illustrated in FIG. 5A, which shows the top view of the sensing device 300, the light source 320A can be disposed at a first location on the support structure 310 (e.g., a bottom left corner of the support structure), and the light source 320B can be disposed at a second location on the support structure 310. The positions of the light sources 320 enable sensor readings to be captured via different paths, as further described herein.

The sensors 330 (e.g., sensor 330A, 330B, and/or 330C) can be photodetectors and/or photosensors configured to sense, detect, and/or measure light (e.g., generated by a light source 320 and reflected by tissue). In some embodiments the sensors 330 can be photodiodes. The sensors 330 can be disposed on the support component 310 as an independent component or as part of a multi-chip package, as described above. The sensors 330 can be disposed on the support structure 310 in different locations, e.g., such that each sensor 330 facilitates the capture of light signals via different light paths.

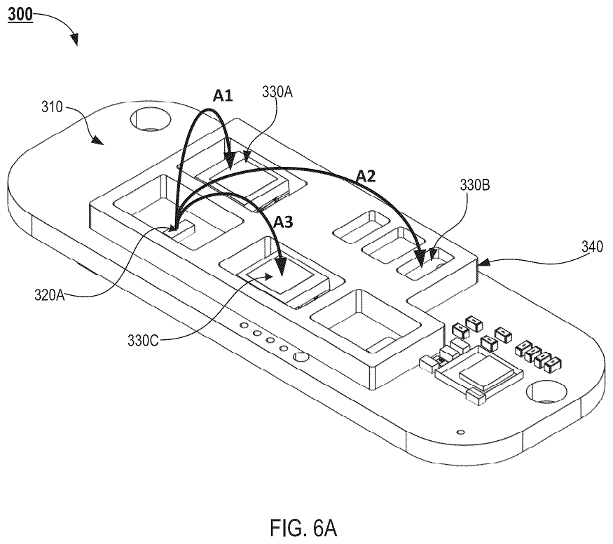

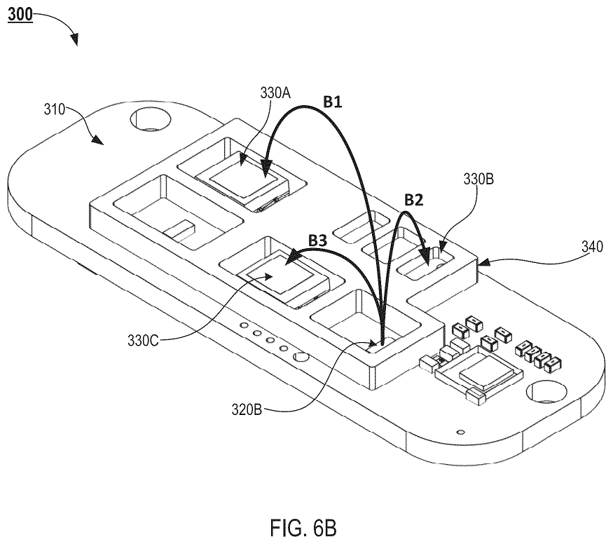

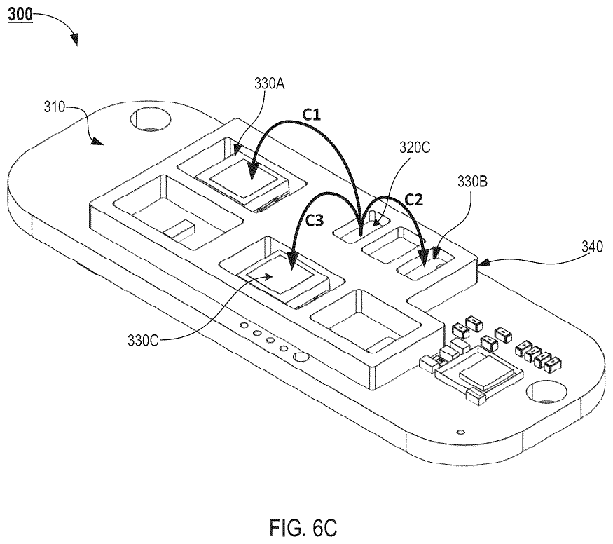

FIGS. 6A-6C show that the three light sources 320 (e.g., 320A, 320B, and 320C) and the three sensors 330 (e.g., 330A, 330B, and 330C) generate nine substantially different light paths for capture patient physiological information (e.g., PPG signals). FIG. 6A shows that the light source 320A can pair with each of three different sensors 330A, 330B, 330C to generate three substantially different and/or distinct light paths A1, A2, and A3 (represented by arrows from the light source 320A to the sensor and/or detector 330A, 330B and 330C, respectively). In particular, light generated by the light source 320A can travel following path A1, which starts at the light source 320A, travels through tissue, and is reflected and captured by the sensor 330A. Light generated by the light source 320A can also travel following path A2, which starts at the light source 320A, travels through tissue, and is reflected and captured by the sensor 330B. Light generated by the light source 320A can also travel following path A3, which starts at the light source 320A, travels through tissue, and is reflected and captured by the sensor 330C. The target tissues and/or regions of the individual seated on the toilet associated with paths A1, A2, A3 can be substantially different and/or distinct from each other. In that way, the sensing device 300 can capture multiple PPG signals, with each PPG signal being directed to a different target tissue and/or region and involving a different light path. Consequently, the sensing device 300 can provide multiple readings, e.g., which can reduce and/or minimize noise, reading errors, and/or other variability across individuals and/or provide more comprehensive information about an individual.

FIG. 6B shows that the light source 320B can pair with each of three different sensors 330A, 330B, 330C to generate three substantially different and/or distinct light paths B1, B2, and B3 (represented by the arrows from the light source 320B to the sensor and/or detector 330A, 330B and 330C, respectively). In particular, light generated by the light source 320B can travel following path B1, which starts at the light source 320B, travels through tissue, and is reflected and captured by the sensor 330A. Light generated by the light source 320B can also travel following path B2, which starts at the light source 320B, travels through tissue, and is reflected and captured by the sensor 330B. Light generated by the light source 320B can also travel following path B3, which starts at the light source 320B, travels through tissue, and is reflected and captured by the sensor 330C. The target tissues and/or regions of the individual seated on the toilet associated with paths B1, B2, and B3 can be substantially different and/or distinct from each other, and from the target tissues and/or regions associated with the paths A1, A2, and A3 described above.

FIG. 6C shows that the light source 320C can pair with each of three different sensors 330A, 330B, 330C to generate three substantially different and/or distinct light paths C1, C2, and C3 (represented by the arrows from the light source 320C to the sensor and/or detector 330A, 330B and 330C, respectively). In particular, light generated by the light source 320C can travel following path C1, which starts at the light source 320C, travels through tissue, and is reflected and captured by the sensor 330A. A light signal generated by the light source 320C can also travel following path C2, which starts at the light source 320C, travels through tissue, and is reflected and captured by the sensor 330B. Similarly, a light signal generated by the light source 320C can also travel following path C3, which starts at the light source 320C, travels through tissue, and is reflected and captured by the sensor 330C. The target tissues and/or regions of the individual seated on the toilet associated with paths C1, C2, and C3 can be substantially different and/or distinct from each other, and from the target tissues and/or regions associated with the paths A1, A2, A3, B1, B2, and B3, described above.

In some embodiments, the locations and/or positions of the light sources 320 and the sensors 330 can also include different heights with respect to each other. For example, as shown in FIG. 5B, the vertical height of the light sources 320A and 320B and the sensors 330A, 330B, and 330C are different. In particular, the height of the light source 320A, defined with respect to the surface 311, is less than the height of the detector 330A, defined with respect to the same surface 311. Similarly, the height of the light source 320B, defined with respect to the surface 311, is less than the height of the detector 330B defined with respect to the same surface 311. The differences in relative height between light sources 320 and sensors and/or detectors 330 can also contribute to generating different and/or distinct light paths (e.g., light paths A1-C3).

The partitioning element 340 can be one or more suitable components disposed on the support structure 310 surrounding the light source(s) 320 and the sensor(s) 330. The partitioning element 340 can function as light-blocking structure configured to prevent light from the light source(s) 320 from saturating the sensor(s) 330. In some embodiments the partitioning element 340 can be configured to direct light toward a target tissue of a subject, or a desired sensor 320 (e.g., after being reflected by the target tissue). In some embodiments, the partitioning element(s) 340 can prevent the light from propagating in an unwanted direction (e.g., escaping and/or leaking from the sensing device 300 horizontally through the cover 350 prior to reaching the target tissue and after being reflected by the target tissue), and instead restricts the direction of propagation of the light towards the desired direction and/or target. For example, the partitioning element(s) 340 can be configured to direct light generated by the light source(s) 320 toward the tissue of an individual, such that the generated light penetrates through the tissue before being reflected back and being captured by at least one sensor 330. Said in other words, the partitioning element(s) 340 can be configured to reduce lateral leakage of light (e.g., electromagnetic radiation) from the light source(s) 320 to the sensor(s) 330 such that the light and/or electromagnetic radiation measured by the sensor(s) 330 is substantially light and/or electromagnetic radiation generated by the light source(s) 320 and reflected by the tissue of a subject, when the subject is seated on the toilet. In some embodiments, the sensor(s) 330 may be disposed in the PPG sensing device 300 to measure a portion of the electromagnetic radiation generated by the light source(s) 320 of the PPG sensing device 300, wherein the portion of the electromagnetic radiation corresponds to electromagnetic radiation reflected by tissue of a subject, when the subject is seated on the toilet. In such embodiments, the partitioning element(s) 340 may be disposed about the light source(s) 320 and the sensor(s) 330 to prevent electromagnetic radiation other than the portion of electromagnetic radiation from being detected by the sensor(s) 330. As shown in FIGS. 4 and 5, the partitioning component 340 can be implemented as a gasket comprising multiple openings. The openings of the partitioning component 340 can be sized and shaped to surround one or more components of the sensing device 300, including the light sources 320 and the sensors 330. More specifically, the openings of the partitioning component 340 can provide enclosures around the light sources 320 and the sensors 330 that limit or block light from propagating in directions away from the cover 350 and tissue of an individual seated above the cover (e.g., on a toilet seat).

In some embodiments, the partitioning element 340 can include multiple portions that can be coupled and/or assembled together, e.g., to isolate the light sources 320 and the sensors 330. That is, in some embodiments, the partitioning element 340 can be modular. Alternatively, in other embodiments, the partitioning element 340 can be made of a monolithic structure. In some embodiments the partitioning element 340 can be made of flexible, elastic materials, including ethylene vinyl acetate (EVA), polyethylene, polyurethane, rubber, etc., e.g., to facilitate placement of the partitioning element 340 tightly between the support component 310 and the cover 350.

The cover 350 can be a panel, shield, casing, lid, or the like that can protect the various components of the sensing device 300 while allowing light to travel from the light sources 320 to the sensors 330. In some embodiments, the cover 350 can transparent and/or translucent. For example, the cover 350 can be made of one or more materials that do not absorb visible and/or IR light generated by the light sources 320. In some embodiments, the cover 350 can be made of materials that absorb negligible amounts of visible and/or IR light such as, for example, quartz, Germanium (Ge), Zinc Selenide (ZnSe), sapphire phase alumina (Al₂O₃), calcium fluoride (CaF₂), potassium bromide (KBr), Barium Fluoride (BaF₂), Silicon (Si), gallium Arsenide (GeAs), and/or polymeric materials such as poly methyl methacrylate (PMMA), polyurethane, polycarbonate, polystyrene, and the like.

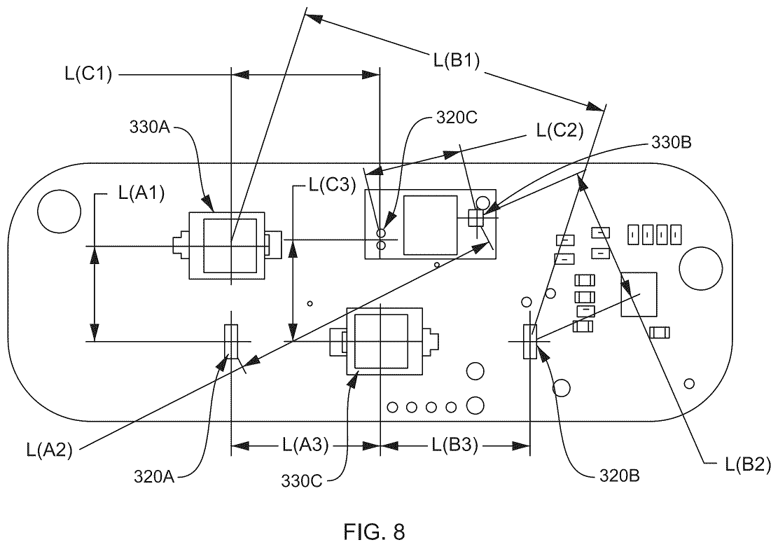

FIG. 8 shows a top view of the sensing device 300 displaying the distances that separate the light sources 320 from the sensors 330 along a horizontal plane of the support structure 310. Each of the distances, represented by L, can be associated with the respective path lengths A1-C3, as described above with reference to FIGS. 6A-6C. For example, the light source 320A and the sensor and/or detector 330C can be separated by a distance L(A3) measured along the horizontal plane, which is associated with the path A3; the light source 320B and the sensor 330C can be separated by a distance L(B3) measured along the horizontal plane, which is associated with the path B3; and so on. While the distances L(A1)-L(C3) are associated with the respective paths A1-C3, the distances are not equal to the path lengths travelled by the light along each path. In particular, the light travelling along each path follows a curved or angled trajectory. Nevertheless, each of the distances L(A1)-L(C3) can provide a relative approximation of the path length travelled by the light along each path. For example, the path length of A3, associated with L(A3), can be less than the path length of B1), associated with L(B1). With more particularity, each of the distances L(A1)-L(C3) can represent a fraction and/or portion of the length of the respective light paths A1-C3. For example, the distance L(A3) represents a linear portion and/or fraction of the total path length A3. In other words, the distance L(A3) represents the fraction of the pathway A3 that light travels parallel to the horizontal plane of the support structure 310. The total magnitude of the path length A3 comprises the distance L(A3) plus the distance that the light travels out of the sensing device 300 (e.g., angled relative to the horizontal plane) to a target tissue and/or region of a subject seated on the toilet, and then reflected from the target tissue and/or region of the subject back into the sensing device 300.

In some embodiments, L(A1)-L(C3) can be between about 1 mm and about 50 mm, including all sub-ranges and values therebetween. For example, L(A1), L(C2), and L(C3) can be between about 1 mm and about 10 mm, including all sub-ranges and values therebetween, including, for example, between about 5 mm and about 7 mm. L(A3), L(B2), L(B3), and L(C1) can be between about 5 mm and about 15 mm, including all sub-ranges and values therebetween, including, for example, between about 8 mm and about 10 mm. L(A2) and L(B1) can be between about 10 mm and about 25 mm, including all sub-ranges and values therebetween, including, for example, between about 16 mm and about 20 mm. As described above, the different path lengths and path locations can enable cleaner and more comprehensive capture of PPG data.

In some embodiments, the PPG sensing devices or systems described herein can implement algorithms for separating the DC and AC components of a PPG signal. For example, as depicted in FIG. 11, systems and devices described herein can implement a method 1100 for dynamic DC level control. The method 1100 can be implemented by a processor, including, for example, an onboard processor of a PPG sensing device (e.g., processor 1004) or a process of a remote compute device to which the PPG sensing device is coupled. The method 1100 can provide a greater dynamic range for capturing the AC component of the PPG signals described herein. As described above, a PPG signal can have two components: a DC component and an AC component. While both components are important, the DC component can be significantly stronger than the AC component. As such, it can be desirable to separate the DC component from the AC component, e.g., using method 1100.

The method 1100 can include receiving or obtaining x sets of readings from the PPG sensors (e.g., PPG sensors 130, 130′, 330, etc.) at a low gain, where x=1, 2, 3, . . . n, at 1102. The processor can average the x sets of low-gain readings, at 1104. This average of the low-gain readings can be representative of or a close approximation of the DC component of the PPG readings. In some embodiments, the processor can set the offset to be subtracted from the PPG readings (I_{OFFSET}) to the average of the low-gain readings, at 1106. Alternatively or additionally, the processor can adjust one or more other parameters, including, for example, current (I_{LED}) delivered to one or more light sources (e.g., light sources 120, 120′, 320, etc.) and therefore the intensity of light generated by the light sources and/or a gain (G_{TIA}) of a transimpedance amplifier in the circuitry that processes the PPG signals, based on the average of the low-gain readings, to ensure capture of a clean AC component of the PPG signal.

At 1108, the method 1100 can include obtaining additional readings from the PPG sensors, e.g., for a predetermined period of time. The predetermined period of time can be, for example, between about a few seconds (e.g., about 5 seconds) and about a few minutes (e.g., about 10 minutes), including all sub-ranges and values therebetween. The additional readings can be processed, e.g., using circuitry such as that depicted in FIG. 12. The processing can include subtracting or removing the I_{OFFSET} from the PPG readings, and then processing the signals using a transimpedance amplifier (TIA) set to a high gain, e.g., to capture the AC component of the PPG readings.

In some embodiments, the processor can average y high-gain readings, where y=1, 2, 3, . . . n, at 1110. At 1112, if the average of the high-gain readings is saturated or high (e.g., outside of a predetermined range or about a predetermined threshold), then the processor can average an additional z sets of low-gain readings to estimate or determine a new DC offset (e.g., DC component of the PPG signal). The new DC offset can then be used to set one or more of I_{OFFSET}, I_{LED}, or G_{TIA}, at 1116. If the average of the high-gain readings is within acceptable ranges (e.g., within a predetermined range or below a predetermined threshold), then the process can continue collecting additional PPG readings, at 1108. In some embodiments, the measurements can continue until the individual or subject is no longer seated, e.g., at the toilet or other seat. In some embodiments, the measurements can continue for a predetermined period of time before the process ends.

In some embodiments, a PPG sensing device can include multiple sensors (e.g., sensors 130, 130′, 330, etc.), and the method 1100 can be implemented for each of the sensors.

FIG. 12 depicts an example circuit for processing PPG readings or signals captured by PPG sensing devices as described herein, according to embodiments. As depicted in FIG. 12, one or more sensors implemented as photodiodes PD (e.g., similar to sensors 130, 130′, 330, etc. as described herein) can capture PPG readings or signals. An input multiplexer MUX can switch in different signals from the photodiodes PD to a transimpedance amplifier TIA. For each photodiode PD, the signal from the photodiode PD (represented by I_{IN}) can be offset by I_{OFFSET}, and the net signal (I_{IN}−I_{OFFSET}) can be input into the transimpedance amplifier TIA. The transimpedance amplifier TIA can apply a gain (G_{TIA}) to the signal from the photodiode PD (e.g., based on a value of resistance R_{F} and/or other parameters), and the resulting output signal V_{OUT} can be used to determine or approximate an AC component of a subject's PPG signal. As described with respect to FIG. 11, one or more of I_{OFFSET}, I_{LED}, or G_{TIA} can be adjusted, e.g., to adjust the output signal.

While a single MUX and a single TIA are depicted in FIG. 12, it can be appreciated that one or more additional MUX or TIA can be used. For example, in some embodiments, a MUX can switch in one or more PD signals to two or more TIA, e.g., for further processing. In some embodiments, a circuit for processing PPG readings can exclude and/or omit the use of an input multiplier MUX, and instead the signals generated by each photodiode PD are sent to a different TIA (e.g., one TIA per each photodiode PD). In some embodiments, once the PD signals are processed by the TIA, the signals can be further processed, e.g., averaged, adjusted (e.g., based on an offset), etc.

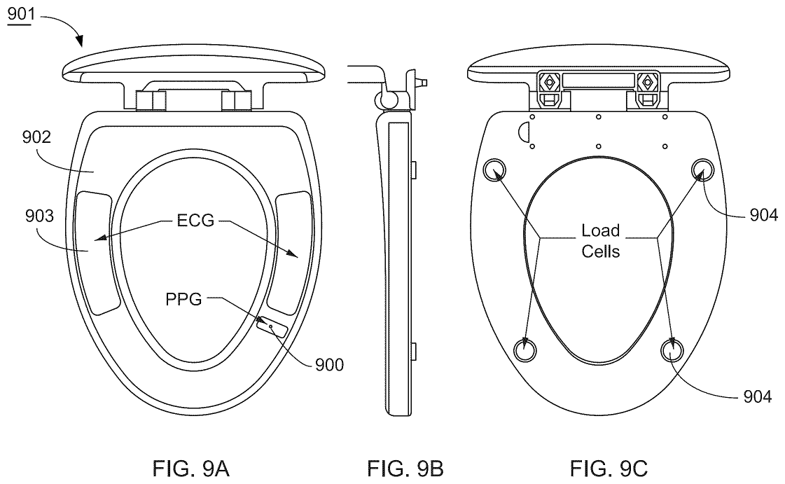

FIGS. 9A-9C show a top, side, and bottom view, respectively, of a sensing device or system (e.g., such as those described with reference to FIGS. 1-8) for monitoring signals associated with various physiological data or conditions of an individual implemented into a toilet seat 901. The toilet seat 901 can include a ring 902. The ring 902 can be a component of the toilet on which an individual, a user and/or subject sits. The ring 902 can be positioned on top of a base (e.g., a top portion of a toilet bowl), such that the ring defines a centrally disposed opening therethrough, e.g., for receiving bodily fluids, defecation, etc. The shape and/or the dimensions of the ring 902 can be substantially similar or correspond to the shape and/or dimensions of the base. For example, the shape of the ring 902 can be circular, oval, elliptical, and/or any other annular shape that generally matches or corresponds to the shape of the base.

The ring 902 can include multiple sensing devices including, for example, a PPG sensing device 900, an ECG sensing device 903, and a BCG sensing device 904. The PPG sensing device 900, which can also be referred to herein as the “sensing device 900” or the “device 900,” can be the same or similar in form and/or function to the sensing devices 100, 100′, and 300 described above with reference to FIGS. 1A-8. For example, the sensing device 900 can include one or more components that are the same or substantially the same as other sensing devices described herein, including as a support structure, one or more light sources, one or more sensor(s) and/or detector(s), one or more partitioning element(s), and/or a cover.

After the ring 902 is installed on a toilet (e.g., using a coupler), the sensing devices included and/or incorporated in the ring 902 can be configured to measure multiple signals present on the ring 902, e.g., when an individual is seated on the ring 902 (e.g., PPG signals, ECG signals, and loads and/or forces associated with the seated individual). The signals can be transmitted to a processor (e.g., of a user device 1060 and/or a compute device 1050, as described with reference to FIG. 10) for processing and/or analysis. In some embodiments, a user can modify an existing toilet to have sensing functionality with toilet seat 901, e.g., by replacing a ring of the toilet with the ring 902. Alternatively, the ring 902 can be provided with other components for installing in a new toilet.

In some embodiments, the PPG sensing devices described herein can be operatively coupled to one or more other devices, e.g., sensing devices and/or compute devices, for monitoring one or more physiological conditions of an individual. FIG. 10 shows a block diagram illustrating a PPG sensing system 1000 in communication with other devices via a network 1005. In some embodiments, the PPG sensing system 1000 can be configured to measure physiological data or conditions, such as, for example, volumetric blood flow variations of an individual seated on the toilet, which can be estimated and/or determined from light signals (e.g., PPG signals). The sensing system 1000 can include component(s) that are structurally and/or functionally similar to those of other sensing systems and devices described herein, including, for example, the sensing device(s) 100, 100′, 300, 900. For example, the sensing system 1000 can include one or more sensor(s) and/or detector(s) 1002 that can be configured to measure PPG signals or other light signal readings. The sensor(s) 1002 can be integrated into or coupled to a ring (e.g., ring 902 shown in FIG. 9) of a toilet to collect sensor data representative of PPG signals of an individual seated on the ring.

In some embodiments, the sensing system 1000 can optionally communicate with complementary sensing system(s) 1070 via a network 1005. The complementary sensing system(s) 1070 can be configured to measure physiological data or signals associated with the same individual as the sensing system 1000. For example, an individual can be seated on a toilet seat, and the sensing system 1000 can measure PPG signals associated with volumetric blood flow variations of the individual, and the complementary sensing system 1070 integrated into the toilet seat and/or operably coupled to the toilet seat can measure an ECG signal, a BCG signal, a temperature of urine or feces, a total weight and/or a partial weight of the individual, etc.

While not depicted, the complementary sensing system(s) 1070 can include one or more sensors, communication interfaces, and/or processors for measuring and/or processing data associated with a seated individual. In some embodiments, the complementary sensing system 1070 can be configured to receive data (e.g., PPG data) from the sensing system 1000, and an onboard processor of the complementary sensing system 1070 can be configured to process and/or analyze this data, e.g., in combination with other data collected by the complementary sensing system 1070, to determine information such heart rate, heart rate variability, left ventricular ejection time, pre-ejection period, flow velocity, pulse transit time (e.g., based on PPG, ECG or BCG data), blood pressure, cardiac output, cardiac contractility, abnormal heart function, blood oxygenation levels (e.g., SpO2), respiration rate, stress levels (e.g., via heart rate variability), body weight (partial and/or total), posture, cardiac waveform characteristics (e.g., magnitudes and/or intervals), internal body temperature, and/or core body temperature of the individual seated on the toilet. In some embodiments, the complementary sensing system(s) 1070 can include a sensing system that is integrated into a toilet seat, as described in the '658 patent, International Patent Application Number PCT/US2022/024236 entitled, “Systems, Devices, and Methods for Monitoring Loads and Forces on a Seat,” filed Apr. 11, 2022 (“the '236 application”), International Patent Application Number PCT/US/2022/029646 entitled Systems, Devices, and Methods for Measuring Body Temperature of a Subject Using Characterization of Feces and/or Urine,” filed May 17, 2022 (“the '646 application”), and International Patent Application Number PCT/US2022/028787 entitled Systems, Devices, and Methods for Measuring Loads of a Seated Subject using Scale Devices,” filed May 11, 2022 (“the '787 application”). The disclosures of each of the foregoing applications are incorporated herein by reference in their entirety.

Sensing system 1000 can communicate with a compute device 1050, one or more user device(s) 1060, one or more third-party device(s) 1090, etc., via a network 1005. The network 1005 can include one or more network(s) that may be any type of network (e.g., a local area network (LAN), a wide area network (WAN), a virtual network, a telecommunications network) implemented as a wired network and/or wireless network and used to operatively couple to any compute device, including PPG sensing system 1000, compute device 1050, user device(s) 1060 and third-party device(s) 1090.

Optionally, the sensing system 1000 can be configured to send data measured by sensor(s) 1002 via a communication interface 1003 to the complementary sensing system(s) 1070, the compute device 1050, one or more user device(s) 1060, and/or one or more third-party device(s) 1090. In some embodiments, the sensing system 1000 can include onboard processing, such as, for example, a processor 1004 implemented as a microprocessor, to process sensor data (e.g., filter, convert, etc.) prior to sending the sensor data to the complementary sensing system(s) 1070, compute device 1050, one or more user device(s) 1060, and/or one or more third-party device(s) 1090. Alternatively, sensing system 1000 can be configured to send raw sensor data to the complementary sensing system(s) 1070, the compute device 1050, one or more user device(s) 1060, and/or one or more third-party device(s) 1090. In some embodiments, processor 1004 can be configured to analyze the sensor data and/or determine information such as volumetric blood, heart rate, or other physiological data or conditions of a subject (e.g., an individual seated on a toilet). In some embodiments, processor 1004 can be configured to present this information to a user, e.g., via an onboard display, audio device, or other output device. In some embodiments, the processor 1004 can interface with the communication interface 1003 to transmit information to another device (e.g., complementary sensing system 1070, user device 1060, compute device 1050, or third-party device 1090) for presenting information to a user. The communication interface 1003 can be configured to allow two-way communication with an external device, including, for example, the compute device 1050, one or more user device(s) 1060, and/or one or more third-party device(s) 1090. The communication interface 1003 can include a wired or wireless interface for communicating over the network 1005.

The compute device 1050 can be configured to process and/or analyze the sensor data, e.g., received from the sensor(s) 1002. In some embodiments, the compute device 1050 can be a nearby compute device (e.g., a local computer, laptop, mobile device, tablet, etc.) that includes software and/or hardware for receiving the sensor data and processing and/or analyzing the sensor data. In some embodiments, the compute device 1050 can be a server that is remote from the sensing system 1000 but can communicate with the sensing system 1000 via network 1005 and/or via another device on the network 1005 (e.g., a user device 1060). For example, sensing system 1000 can be configured to transmit sensor data to a nearby device (e.g., a complementary sensing system 1070 or a user device 1060), e.g., via a wireless network (e.g., Wi-Fi, Bluetooth®, Bluetooth® low energy, Zigbee and the like), and then that device can be configured to transmit the sensor data to the compute device 1050 for further processing and/or analysis.

The user device(s) 1060 can be compute device(s) that are associated with a user of a toilet equipped with the sensing system 1000. Examples of user device(s) 1060 can include a mobile phone or other portable device, a tablet, a laptop, a personal computer, a smart device, etc.). In some embodiments, a user device 1060 can receive sensor data from the sensing system 1000 and process that sensor data before passing the sensor data to the compute device 1050. For example, a user device 1060 can be configured to reduce noise (e.g., filter, time average, etc.) raw sensor data. In some embodiments, a user device 1060 can be configured to analyze the sensor data and present (e.g., via a display) information representative of or summarizing the sensor data. In some embodiments, a user device 1060 can provide weight information, body temperature information, heart rate information, etc. to a user. In some embodiments, a user device 1060 can transmit the sensor data to the compute device 1060, which can analyze the sensor data and send information representative of or summarizing the sensor data back to the user device 1060 for presenting (e.g., via a display) to a user.

The third-party device(s) 1090 can be compute device(s) associated with other individuals or entities that have requested and/or been provided access to a user's data. For example, the third-party device(s) 1090 can be associated with healthcare professionals (e.g., physicians, nurses, therapists) and/or caregivers of the user. The user can select to have certain third parties have access to the user's health data (e.g., including health data obtained from sensor data collected by sensing system 1000). The third parties can then track the user's health information to determine whether the user is at risk for certain conditions and/or needs certain interventions, treatments, or care.

The compute device 1050 can include a processor 1052, a memory 1054, and an input/out device (I/O) 1056 (or a multiplicity of such components). The memory 1054 can be, for example, a random-access memory (RAM), a memory buffer, a hard drive, a database, an erasable programmable read-only memory (EPROM), an electrically erasable read-only memory (EEPROM), a read-only memory (ROM), and/or so forth. In some embodiments, the memory 1054 stores instructions that cause processor 1052 to execute modules, processes, and/or functions associated with processing and/or analyzing sensor data from sensing system 1000.

The processor 1052 of compute device 1050 can be any suitable processing device configured to run and/or execute functions associated with processing and/or analyzing sensor data from sensing system 1000. For example, processor 1052 can be configured to process and/or analyze sensor data (e.g., received from sensor(s) 1002), to determine a volumetric blood flow, heart rate, or other physiological data or conditions of an individual. The processor 1052 can be a general-purpose processor, a Field Programmable Gate Array (FPGA), an Application Specific Integrated Circuit (ASIC), a Digital Signal Processor (DSP), and/or the like.

The I/O device 1056 of the compute device 1050 can include one or more components (e.g., a communication or network interface) for receiving information and/or sending information to other devices (e.g., sensing system 1000, user device(s) 1060, third-party device(s) 1090). In some embodiments, the I/O device 1056 can optionally include or be operatively coupled to a display, audio device, or other output device for presenting information to a user. In some embodiments, the I/O device 1056 can optionally include or be operatively coupled to a touchscreen, a keyboard, or other input device or receiving information from a user.

While complementary sensing system(s) 1070, user device(s) 1060, and third-party-device(s) 1090 are not depicted with any onboard memory, processing, and/or I/O devices, it can be appreciated that any one of these devices can include components (e.g., a memory, a processor, a I/O device, etc.) that enable it to perform functions such as, for example, processing and/or analyzing the sensor data, or using the sensor data to determine physiological information about an individual (e.g., PPG, weight, body core temperature, BCG, posture, impedance, etc.)

It should be understood that the disclosed embodiments are not representative of all claimed embodiments. As such, certain aspects of the disclosure have not been discussed herein. That alternate embodiments may not have been presented for a specific portion of the innovations or that further undescribed alternate embodiments may be available for a portion is not to be considered a disclaimer of those alternate embodiments. Thus, it is to be understood that other embodiments can be utilized, and functional, logical, operational, organizational, structural and/or topological modifications may be made without departing from the scope of the disclosure. As such, all examples and/or embodiments are deemed to be non-limiting throughout this disclosure

Some embodiments described herein relate to methods. It should be understood that such methods can be computer implemented methods (e.g., instructions stored in memory and executed on processors). Where methods described above indicate certain events occurring in certain order, the ordering of certain events can be modified. Additionally, certain of the events can be performed repeatedly, concurrently in a parallel process when possible, as well as performed sequentially as described above. Furthermore, certain embodiments can omit one or more described events.

As used in this specification and/or any claims included herein the singular forms “a,” “an,” and “the” include plural referents unless the context clearly dictates otherwise. Thus, for example, the term “a member” is intended to mean a single member or a combination of members, “a material” is intended to mean one or more materials, and/or the like.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one implementation, to at least one, optionally including more than one, A, with no B present (and optionally including elements other than B); in another implementation, to at least one, optionally including more than one, B, with no A present (and optionally including elements other than A); in yet another implementation, to at least one, optionally including more than one, A, and at least one, optionally including more than one, B (and optionally including other elements); etc.

As used herein, the term “set” can refer to multiple features or a singular feature with multiple parts and/or components. For example, when referring to a set of light sources, the set of light sources can be considered as one single light source with multiple components (e.g., a lens, case reflective cavity, diode, etc.), or the set of light sources can be considered as multiple, distinct light sources.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

1. An apparatus, comprising:
a support structure disposed in a seat of a toilet;
a plurality of light sources disposed on the support structure and configured to generate electromagnetic radiation that can be delivered to tissue of a subject seated on the toilet, the plurality of light sources including at least two types of light sources;
a sensor disposed on the support structure, the sensor configured to measure electromagnetic radiation; and
a partitioning element disposed about the plurality of light sources and the sensor, the partitioning element configured to reduce lateral leakage of electromagnetic radiation from the plurality of light sources to the sensor such that the electromagnetic radiation measured by the sensor is substantially electromagnetic radiation generated by the plurality of light sources and scattered by tissue of the subject when the subject is seated on the toilet; and
a processor operatively coupled to the plurality of light sources and the sensor, the processor configured to:
receive signals from the sensor, the signals associated with different sensing paths between at least one light source from the plurality of light sources and the sensor; and
process the signals to determine a physiological parameter of the subject.

2. The apparatus of claim 1, wherein the at least two types of light sources includes a red light source and an infrared light source.

3. The apparatus of claim 1, wherein the sensor includes a photodiode.

4. The apparatus of claim 1, wherein the partitioning element is a gasket.

5. The apparatus of claim 1, further comprising a cover coupled to the seat, the cover configured to shield the plurality of light sources and the sensor from the tissue of the subject while allowing the electromagnetic radiation generated by the plurality of light sources to pass through.

6. The apparatus of claim 5, further comprising one or more spacers configured to collectively define:
a predetermined distance between the sensor and the cover; and
a predetermined distance between each light source from the plurality of light sources and the cover.

7. The apparatus of claim 1, wherein the signals received from the sensor are indicative of a photoplethysmogram (PPG) of the subject.

8. The apparatus of claim 7, wherein the signals received from the sensor include:
a first signal associated with a first sensing path between a first light source from the plurality of light sources and the sensor; and
a second signal associated with a second sensing path between a second light source from the plurality of light sources and the sensor.

9. The apparatus of claim 8, wherein the first signal and the second signal are measured sequentially by the sensor.

10. The apparatus of claim 8, wherein the first signal and the second signal are measured simultaneously by the sensor.

11. A system, including:
a seat of a toilet;
a support structure disposed on the seat of the toilet;
a plurality of light sources disposed on the support structure and configured to generate electromagnetic radiation that can be delivered to tissue of a subject seated on the toilet, the plurality of light sources including at least two types of light sources;
a plurality of sensors disposed on the support structure, the plurality of sensors configured to measure electromagnetic radiation;
a partitioning element disposed about the plurality of light sources and the plurality of sensors, the partitioning element configured to reduce lateral leakage of electromagnetic radiation from the plurality of light sources to the plurality of sensors such that the electromagnetic radiation measured by the plurality of sensors is substantially electromagnetic radiation generated by the plurality of light sources and scattered by tissue of the subject; and
a processor operatively coupled to the plurality of light sources and the plurality of sensors, the processor configured to:
receive signals from each sensor of the plurality of sensors, the signals received from each sensor of the plurality of sensors associated with different sensing paths between at least one light source from the plurality of light sources and that sensor; and
process the signals to determine a physiological parameter of the subject.

12. The system of claim 11, wherein the support structure is integrated into the seat of the toilet.

13. The system of claim 11, further comprising a cover coupled to the support structure, the cover configured to shield the plurality of light sources and the plurality of sensors from the tissue of the subject while allowing the electromagnetic radiation generated by the plurality of light sources to pass through.

14. The system of claim 13, further comprising one or more spacers configured to collectively define:
a predetermined distance between the each sensor from the plurality of sensors and the cover; and
a predetermined distance between each light source from the plurality of light sources and the cover.

15. The system of claim 11, wherein the at least two types of light sources include a red light source and an infrared light source.

16. The system of claim 11, wherein the plurality of sensors includes at least one photodiode.

17. The system of claim 11, wherein the plurality of light sources includes a first, a second, and a third light source, and the plurality of sensors includes a first, a second, and a third sensor, the processor being configured to receive signals from each sensor of the plurality of sensors by:
receiving three signals from the first sensor, each of the three signal from the first sensor associated with a sensing path between one of the first, the second, or the third light source and the first sensor;
receiving three signals from the second sensor, each of the three signal from the second sensor associated with a sensing path between one of the first, the second, or the third light source and the second sensor; and
receiving three signals from the third sensor, each of the three signal from the third sensor associated with a sensing path between one of the first, the second, or the third light source and the third sensor.

18. The system of claim 17, wherein the three signals received from the first, the second, and the third sensor are indicative of a photoplethysmogram (PPG) of the subject.

19. The system of claim 18, wherein a subset of the three signals received from the first, the second, and the third sensor are measured sequentially.

20. The system of claim 18, wherein a subset of the three signals received from the first, the second, and the third sensor are measured simultaneously.
